(19) United States
(12) Patent Application Publication
(10) Pub. No.: US 2012/0071823 A1
(43) Pub. Date: Mar. 22, 2012

(54) MEDICAL BALLOON HAVING IMPROVED STABILITY AND STRENGTH

(75) Inventor: John J. Chen, Plymouth, MN (US)

(73) Assignee: BOSTON SCIENTIFIC SCIMED, INC., Maple Grove, MN (US)

(21) Appl. No.: 13/238,082

(22) Filed: Sep. 21, 2011

Related U.S. Application Data
(60) Provisional application No. 61/385,196, filed on Sep. 22, 2010.

(51) Int. Cl. A61L 31/04; B29C 47/20; C08K 5/29

(52) U.S. Cl. 604/96.01; 524/195; 264/209.3

(57) ABSTRACT

A medical device, the medical device formed at least in part from a melt blend of at least one polymer comprising hydrolysable groups and a carbodiimide.

### Cross-Reference to Related Applications

This application is claims priority to U.S. Patent Provisional Application No. 61/385,196 filed Sep. 22, 2010, the entire contents of which are hereby incorporated herein by reference.

### FIELD OF THE INVENTION

The present invention relates to the field of insertable or implantable medical devices including catheter assemblies and expandable medical balloons.

### BACKGROUND OF THE INVENTION

Balloon dilatation catheters having an expandable medical balloon disposed thereon are used in a variety of procedures to open blood vessels or other passageways in the body that may be blocked by obstructions or stenosis including plain old balloon angioplasty (POBA) or percutaneous transluminal coronary angioplasty (PTCA), stent delivery and peripheral catheter procedure.

Dilatation catheters are generally formed from thin, flexible tubing having an inflatable balloon at or near a distal tip of the catheter that can be inflated with fluid that is communicated to the balloon through a lumen of the catheter. In a typical angioplasty procedure, the balloon dilatation catheter is passed through the vasculature to the location of a stenosis in an artery, and the balloon is inflated to a predetermined size and shape to open the blocked artery.

The balloon is typically expanded to a diameter many times that of the uninflated diameter in order to open an obstructed vessel. Desirable balloon properties include strength, softness, flexibility and a thin, low profile which are important for achieving the performance characteristics of folding in an uninflated state, tracking, crossing and recrossing the area of the obstruction or stenosis in a vessel in an uninflated state. Other important properties in the continuing effort to create even thinner, lower profile balloons include burst strength, compliance, and resistance to fatigue along with an ability to track, cross and recross increasingly narrow passages in obstructed vessels.

Polymer materials that have been used for making expandable medical balloons include polyolefins such as polyethylene, polyvinyl chloride, polyesters such as polyethylene terephthalate (PET) and polybutylene terephthalate (PBT) and copolyesters, polyether-polyester block copolymers (e.g. HYTREL® or ARNITEL®), polyamides, polyurethane, poly(ether-block-amide) (PEBAX®) and the like.

One problem that can occur during the manufacturing process with polymers having functional groups such as esters, amides or acid anhydride groups is hydrolysis of the polymer material which can weaken the polymer material due to breakdown of the polymer chains.

There remains a need in the art for balloon materials having enhanced performance.

### SUMMARY OF THE INVENTION

The present invention relates to compositions and methods for making medical devices, particularly catheter assemblies wherein at least a portion of the medical device is formed from a melt blend of at least one polymer which comprises groups that undergo hydrolysis and a carbodiimide.

In one embodiment the medical device is an expandable dilatation balloon.

In one embodiment the expandable dilatation balloon is formed from the melt blend product of at least one poly(ether-block-amide) and a carbodiimide.

These and other aspects, embodiments and advantages of the present invention will become immediately apparent to those of ordinary skill in the art upon review of the Detailed Description and Claims to follow.

### BRIEF DESCRIPTION OF THE DRAWINGS

FIG. 1 is a longitudinal cross-section illustrating an embodiment of a catheter assembly.

FIG. 2 is a perspective view of one embodiment of a dilatation balloon in accordance with the invention.

### DETAILED DESCRIPTION OF THE INVENTION

While this invention may be embodied in many forms, there are described in detail herein specific embodiments of the invention. This description is an exemplification of the principles of the invention and is not intended to limit the invention to the particular embodiments illustrated.

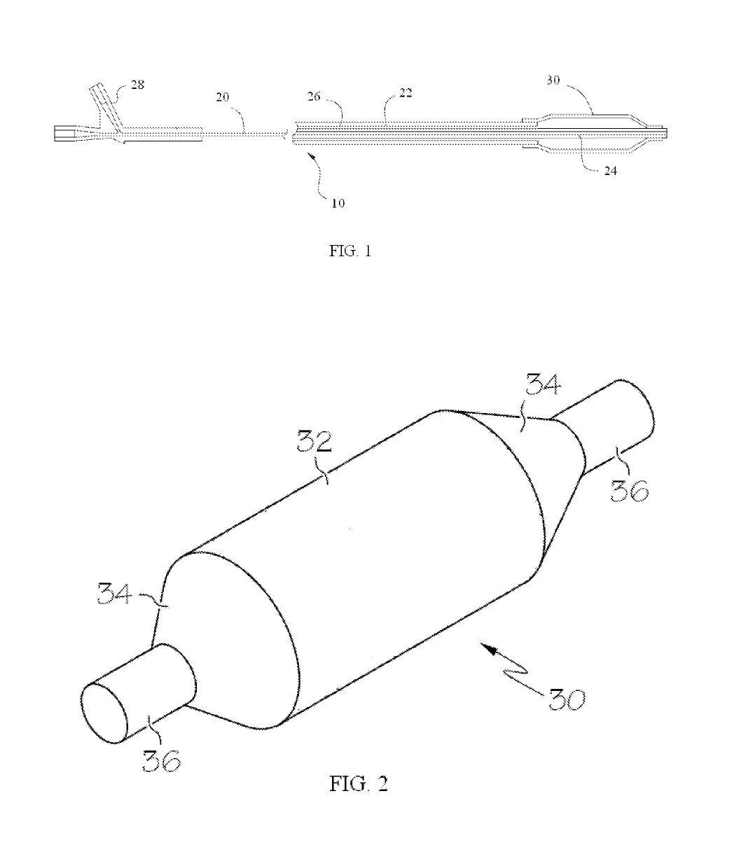

Turning now to the figures, FIG. 1 is a longitudinal cross-sectional view illustrating an embodiment of a balloon catheter assembly 10. Balloon catheter assembly is illustrative of a representative OTW angioplasty balloon catheter. Such balloon catheters are discussed, for example, in commonly assigned U.S. Pat. Nos. 6,113,579, 6,517,515, 6,514,228, each of which is incorporated by reference herein in its entirety. In this embodiment, catheter 10 has an elongate shaft assembly 20 and an expandable balloon member 30 disposed at the distal end thereof. The shaft assembly 20 includes an inner tube 24 and an outer tube 22. Outer tube 22 is coaxially disposed about inner tube 24 to define an annular inflation lumen 26. Manifold assembly 28 is conventional.

Any portion of catheter assembly 10 can be formed from the compositions disclosed herein including inner tube 24, outer tube 22 and expandable balloon 30.

In one embodiment, balloon 30 is formed from the compositions disclosed herein.

FIG. 2 is a perspective view of an expandable dilatation balloon 30 according to the invention. Balloon 30 has body portion 32, cone portions 34 and waist portions 36.

Balloon 30 is formed from a polymer material that comprises functional groups that undergo hydrolysis in the presence of moisture under the right conditions.

For example, balloon 30 is formed from a polymer material comprising ester, amide or acid anhydride functional groups.

Balloon 30 may be formed from polyamides such as nylon 12 available from Degussa-Hüls AG, North America (national headquarters in Düsseldorf, Germany) under the tradename of Vestamid® L2101F (nylon 12 is available from a variety of polymer manufacturers), nylon 6 and nylon 66; polyesters such as polyethylene terephthalate or polybutylene terephthalate; polyether-polyesters such as those sold under the tradename of HYTREL® from DuPont in Wilmington, Del. and those sold under the tradename of ARNITEL® available from DSM Engineering Plastics in Birmingham, Mich.; poly(ether-block-amide) copolymers available from Arkema under the tradename of PEBAX® including PEBAX® 6333, PEBAX® 7033 and PEBAX® 7233, polyester polyurethanes, polycarbonates, polyester carbonates, polyesteramides, polycaprolactones, polylactic acid, polyglycolide, polylactide-go-glycolide, naturally occurring polysaccharides, and mixtures thereof. This list is intended for illustrative purposes only and not as a limitation on the scope of the present invention.

Preferred balloon materials are the poly(ether-block-amide) copolymers.

Because these polymers undergo hydrolysis in the presence of moisture, they are susceptible during the melt extrusion process to a reduction in the size of the polymer chain as a result of the hydrolysis.

It is therefore desirable to add a moisture scavenger to the polymer composition during the extrusion process that will rapidly react with water before the water molecules can attack the functional groups of the polymer chain and prevent molecular weight reduction in the balloon tubing.

For example, either a monomeric carbodiimide or a polymeric carbodiimide can be added to the polymer composition during the melt extrusion process.

These carbodiimides may be aliphatic, cycloaliphatic or aromatic in nature. Suitably, the carbodiimides are aliphatic or cycloaliphatic carbodiimides, and most suitably the carbodiimide is aliphatic

Suitable monomeric carbodiimides are represented by the following general structure.

R-N═C═N—R′

wherein R and R′ are monovalent, R′ may be the same as or different than R and may be independently aromatic, aliphatic, or cycloaliphatic, and may substituted with functional groups.

R and R′, for example, may be independently C₁-C₂₀ alkyl or C₃-C₁₀ cycloalkyl or C₁-C₂₀ alkenyl group, and may be cyclic or branched, or may contain a C₈-C₁₆ aromatic ring, and may be substituted with functional groups. Examples of functional groups include, but are not limited to, cyanato and isocyanato, halo, amido, carboxamido, amino, imido, imino, silyl, etc. These lists are intended for illustrative purposes only and not as a limitation on the scope of the present invention.

Specifically, R and R′ may be independently C₆H₁₂, (CH₂)ₙW wherein n is 1-3, and W may be CH₃, NH₂, NCO, for example.

Specific examples of monomeric carbodiimides useful herein include, but are not limited to, N,N′-dicyclohexylcarbodiimide (DCC), N,N′-diisopropylcarbodiimide or 1-ethyl-3-(3-dimethylaminopropyl) carbodiimide (EDC, EDAC or EDCI), N,N′-diphenylcarbodiimide, N,N′-di-2,6-diisopropylphenylcarbodiimide, etc. See, for example, U.S. Patent Publication No. 2009/0176938, the entire content of which is incorporated by reference herein in its entirety. This list is intended for illustrative purposes only, and not as a limitation on the scope of the present invention.

Commercially available monomeric carbodiimides include those sold by Rhein Chemie in Mannheim, Germany under the tradename of Stabaxol®. One specific example of a monomeric carbodiimide is Stabaxol® I (bis-2,6-diisopropylphenylcarbodiimide).

Suitable polymeric carbodiimides are represented by the following general structure:

RN═C═N—R′ₙ

wherein R is monovalent R′ is divalent, n is 2 to 50, suitably 2 to 45, more suitably 2 to 20 and preferably 5 to 20.

R may be, for example, C₁-C₂₀ alkyl or C₃-C₁₀ cycloalkyl or C₁-C₂₀ alkenyl group, and may be cyclic or branched, or may contain a C₈-C₁₆ aromatic ring, and may be substituted with functional groups. R′ may be a divalent group corresponding to any for the foregoing, e.g., C₁-C₂₀ alkylene, C₃-C₁₀ cycloalkylene, etc. Examples of functional groups include, but are not limited to, cyanato and isocyanato, halo, amido, carboxamido, amino, imido, imino, silyl, etc. These lists are intended for illustrative purposes only, and not as a limitation on the scope of the present invention.

Suitable polymeric carbodiimides useful herein include, for example, repeat units of N,N′-dicyclohexylcarbodiimide, N,N′-diisopropylcarbodiimide, 1-ethyl-3-(3-dimethyl aminopropyl)carbodiirnide hydrochloride, N,N′-diphenylcarbodiimide, N,N′-di-2,6-diisopropylphenylcarbodiimide, 4,4′-dicyclohexylmethanecarbodiimide, tetramethylxylylenecarbodiirnide (aromatic carbodiimide), N,N-dimethylphenylcarbodiimide, N,N′-di-2,6-diisopropylphenylcarbodiimide, 2,2′,6,6′-tetraisopropyl diphenyl carbodiimide (aromatic carbodiimide), aromatic homopolymer of 1,3,5-triisopropyl-2,4-diisocyanatobenzene aromatic heteropolymer of 1,3,5-triisopropyl-2,4-diisocyanatobenzene and 2,6-diisopropyl phenyl isocyanate, or combinations thereof.

See U.S. Pat. Nos. 5,130,360, 5,859,166, 7,368,493, 7,456,137, and U.S. Patent Publication Nos. 2007/0278452 and 2009/0176938, each of which is incorporated by reference herein in its entirety.

Specific examples of R′ include, but are not limited to, divalent radicals derived from 2,6-diisopropylbenzene, naphthalene, 3,5-diethyltoluene, 4,4′-methylene-bis(2,6-diethylenephenyl), 4,4′-methylene-bis(2-ethyle-6-methylphenyl), 4,4′-methylene-bis(2,6-diisopropylephenyl), 4,4′-methylene-bis(2-ethyl-5-methylcyclohexyl), 2,4,6-triisopropylephenyl, n-hexane, cyclohexane, dicyclohexylmethane, and methylcyclohexane, and the like.

Again, aliphatic groups are preferred.

The Stabaxol P series of carbodiimides available from Rhein Chemie in Mannheim, Germany are examples of commercially available aromatic polycarbodiimides.

Isocyanate termination of the polymer chain is one preferred embodiment from the standpoint of stability against hydrolysis under conditions of storage. See, for example, U.S. Patent Publication No. 2009/0318628, the entire content of which is incorporated herein by reference wherein examples or diisocyanates for producing aliphatic, cycloaliphatic and aromatic carbodiimides include 1,5-naphthalene diisocyanate, 4,4′-diphenylmethane diisocyanate, 4,4′-diphenyldimethylmethane diisocyanate, 1,3-phenylenediisocyanate, 1,4-phenylenediisocyanate, 2,4-tolylenediisocyanate, 2,6-tolylenediisocyanate, hexamethylene diisocyanate, cyclohexane-1,4-diisocyanate, xylylene diisocyanate, isophorone diisocyanate, dicyclohexylmethane-4,4′-diisocyanate, methylcyclohexane diisocyanate, tetramethylxylylene diisocyanate, 3,3′,5,5′-tetraisopropylbiphenyl-4,4′-diisocyanate, and 1,3,5-triisopropylbenzene-2,4-diisocyanate. Mixtures of isocyanates may be employed as well. See U.S. Patent Publication No. 2008/0064826 the entire content of which is incorporated by reference herein.

The carbodiimides useful herein react in the presence of water to produce urea the reaction of which is represented by the following general formula:

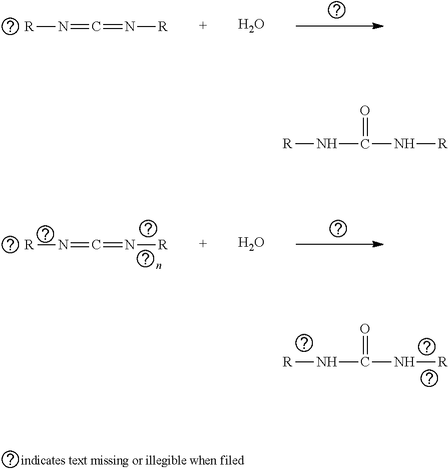

The carbodiimide is useful in amounts of about 10% by weight of the polymer composition or less, suitably about 0.1% to about 10%, more suitably about 0.5% to about 5% and most suitably about 1% to about 2% by weight of the polymer composition.

Both the monomeric and the polymeric forms help to prevent a decrease in the molecular chain size and consequently a decrease in molecular weight, and form a nanocomposite at the molecular level of the tubing and thus reinforce the polymer material.

Whether or not it is catheter tubing or a balloon which is being formed, the carbodiimide is added to the polymer in melt form such as during the extrusion process.

The balloon may be formed using any suitable method known in the art. In some embodiments, the method suitably includes forming a tubular parison, stretching the tubular parison, placing the balloon parison in a balloon mold, and forming a balloon by radially expanding the tubular parison into the balloon mold. The balloon is then heat set. Balloon forming with stretching and radial expansion is disclosed in U.S. Pat. Nos. 5,913,861, 5,643,279 and 5,948,345, and in commonly assigned U.S. Pat. Nos. 6,946,092 and 7,1010,597, each of which is incorporated by reference herein in its entirety.

The description provided herein is not to be limited in scope by the specific embodiments described which are intended as single illustrations of individual aspects of certain embodiments. The methods, compositions and devices described herein can comprise any feature described herein either alone or in combination with any other feature(s) described herein. Indeed, various modifications, in addition to those shown and described herein, will become apparent to those skilled in the art from the foregoing description and accompanying drawings using no more than routine experimentation. Such modifications and equivalents are intended to fall within the scope of the appended claims.

All publications, patents and patent applications mentioned in this specification are herein incorporated by reference in their entirety into the specification to the same extent as if each individual publication, patent or patent application was specifically and individually indicated to be incorporated herein by reference. Citation or discussion of a reference herein shall not be construed as an admission that such is prior art.

1. A medical device, the medical device formed at least in part from a melt blend of at least one polymer comprising hydrolysable groups, and at least one carbodiimide or at least one polycarbodiimide or a mixture thereof.

2. The medical device of claim 1 wherein the medical device is an expandable medical balloon or catheter tubing.

3. The medical device of claim 1 wherein the at least one polymer comprises ester, amide, acid anhydride groups or mixtures thereof.

4. The medical device of claim 1 wherein the melt blend comprises at least one polymer which is a member selected from the group consisting of polyamides, polyesters, polyurethanes, polyether-polyesters, poly(ether-block-amide) copolymers, polyester polyurethanes, polycarbonates, polyester carbonates, polyesteramides, polycaprolactones, polylactic acid, polyglycolide, polylactide-go-glycolide, naturally occurring polysaccharides, and mixtures thereof.

5. The medical device of claim 1 wherein the at least one polymer is a polyamide or a polyester.

6. The medical device of claim 1 wherein the at least one polymer is a poly(ether-block-amide).

7. The medical device of claim 1 wherein the carbodiimide reacts with moisture in the melt blend to form urea.

8. The medical device of claim 1 wherein the carbodiimide is employed in the melt blend in amounts of about 0.5% to about 5% by weight of the polymer composition.

9. The medical device of claim 1 wherein the carbodiimide is employed in the melt blend in amounts of about 1% to about 2% by weight of the polymer composition.

10. The medical device of claim 1 wherein the carbodiimide is aliphatic, cycloaliphatic or aromatic.

11. The medical device of claim 1 wherein the carbodiimide is aliphatic.

12. The medical device of claim 1 wherein the carbodiimide is monomeric having the following general structure:
R—N═C═N—R′

13. The medical device of claim 12 wherein R and R′ are monovalent, R′ may be the same as or different than R and may be independently aromatic, aliphatic, or cycloaliphatic.

14. The medical device of claim 12 wherein R and R′are independently C₁-C₂₀ alkyl, C₃-C₁₀ cycloalkyl or C₁-C₂₀ alkenyl group, and may be cyclic or branched, or may contain a C₈-C₁₆ aromatic ring.

15. The medical device of claim 14 wherein R and R′are independently substituted with a functional group selected from the group consisting of isocyanate, halo, amido, carboxamido, amino, silyl, imido, imino and silyl.

16. The medical device of claim 12 wherein the carbodiimide is bis-2,6-diisopropylphenylcarbodiimide.

17. The medical device of claim 1 wherein the carbodiimide is a polycarbodiimide having the following general structure:
RN═C═N—R′ₙ

18. The medical device of claim 17 wherein n is 2 to 50.

19. The medical device of claim 17 wherein n is 5 to 45.

20. The medical device of claim 17 wherein n 5 to 20.

21. The medical device of claim 17 wherein the polycarbodiimide comprises terminal isocyanate groups.

22. The medical device of claim 17 wherein R is C₁-C₂₀ alkyl, C₃-C₁₀ cycloalkyl, C₁-C₂₀ alkenyl group, and may be cyclic or branched, or may contain a C₈-C₁₆ aromatic ring.

23. The medical device of claim 17 comprising at least one functional group which is a member selected from the group consisting of isocyanato, halo, amido, carboxamido, amino, imido, imino and silyl.

24. The medical device of claim 1 wherein the at least one polycarbodiimide is tetramethylxylylenecarbod iimide.

25. A expandable medical balloon, the medical balloon is formed from a melt blend product of at least one poly(ether-block-amide) copolymer, and at least on carbodiimide, or at least one polycarbodiimide or a mixture thereof.

26. A method of making tubing for a catheter shaft or an expandable medical balloon, the method comprising:
providing a polymer composition in the form of a melt;
blending with said polymer composition at least one carbodiimide or at least one polycarbodiimide or a mixture thereof;
extruding said melt blend in the form of tubing.

27. The method of claim 26 wherein further comprising radially expanding said tubing in a mold to form an expandable medical balloon.
